(19) United States
(12) Patent Application Publication
(10) Pub. No.: US 2017/0136271 A1
(43) Pub. Date: May 18, 2017

(54) PERSONAL AIR FILTRATION DEVICE

(71) Applicant: Jason Munster, Cambridge, MA (US)

(72) Inventor: Jason Munster, Cambridge, MA (US)

(21) Appl. No.: 15/417,575

(22) Filed: Jan. 27, 2017

Related U.S. Application Data
(63) Continuation-in-part of application No. PCT/US2015/042211, filed on Jul. 27, 2015.

(51) Int. Cl. A62B 23/02; B01D 46/42; A61M 16/10; A47D 15/00; A61M 16/06; A61M 16/00; A62B 18/00; A47D 13/04; B01D 46/00; A61M 16/20

(52) U.S. Cl. CPC A62B 23/025; B01D 46/0023; B01D 46/0086; B01D 46/429; B01D 46/4272; B01D 46/0036; B01D 46/4245; A61M 16/106; A61M 16/208; A61M 16/0616; A61M 16/0683; A61M 16/0066; A62B 18/006; A47D 13/04; A47D 15/00; B01D 2267/30; B01D 2279/40; B01D 2265/02; A61M 2202/0216; A61M 2202/0275; A61M 2205/7545; A61M 2205/7563; A61M 2016/0027; A61M 2205/7581; A61M 2209/08; A61M 2205/584; A61M 2205/3592; A61M 2205/3327

(57) ABSTRACT

The invention provides a personal filtration apparatus with a multi-stage filter. In some embodiments each section can be individually replaced for reduced cost

### BACKGROUND OF THE INVENTION

Field of the Invention

The present invention is in the technical field of air filtration. More particularly, the present invention is in the technical field of personal portable air filtration.

Summary of the Related Art

Over 2 billion people worldwide live in hazardous air pollution. Current filters are either stationary room filtration apparatuses that are not portable, and thus of little use outdoors in polluted areas, or masks. Current masks only filter PM2.5, one of the three major life-threatening common air pollutants, and are very costly and out of the price range of those most affected by air pollution. Moreover, the only commercial masks capable of effectively filtering SO₂ and NO₂, the two remaining deadly air pollutants, are full-face gas masks with large chemical-based scrubbers that are expensive and uncomfortable. This renders current affordable masks only able to filter out PM2.5, leaving them incapable of filtering other major pollutants that are proven to cause health issues and early death. Further, many masks do not get an effective seal against the face, allowing outside air to pass around the filter and instead go through the leaks. Further, nearly all solutions require sufficient design and material to make them not cost-effective for most of the world's citizens. U.S. Pat. No. 6,752,149 discloses a nasal air filter with replaceable filters which must form a tight seal on the face around the nose. There is, therefore, a need for inexpensive personal air filters that can form a good seal with the mouth and filter out a variety of air pollutants.

### BRIEF SUMMARY OF THE INVENTION

The present invention is a multi-inlet mouth-held filtration apparatus, face mask, baby stroller attachment, or portable filtration device with a multi-stage filter to inexpensively filter out many different particulates or chemicals depending on the specific environment.

### BRIEF DESCRIPTION OF THE DRAWINGS

FIG. 1 is a top view of an embodiment of a multi-inlet filtration apparatus of the present invention.

FIG. 2 is a perspective view of an embodiment of a of the multi-inlet filtration apparatus of the present invention.

FIG. 3 is a perspective view of an embodiment of a multi-stage filter of the present invention.

FIG. 4 is an exploded view of an embodiment of a multi-stage filter of the present invention.

FIG. 5 is a perspective view of an embodiment of a mask filtration apparatus of the present invention.

FIG. 6 is a top view of an embodiment of a portable personal fan-powered filtration device to connect to a baby stroller or for other personal filtration

FIG. 7 shows filtration efficiency results for a PM2.5 multi-layered polyester filter.

### DETAILED DESCRIPTION OF THE PREFERRED EMBODIMENTS

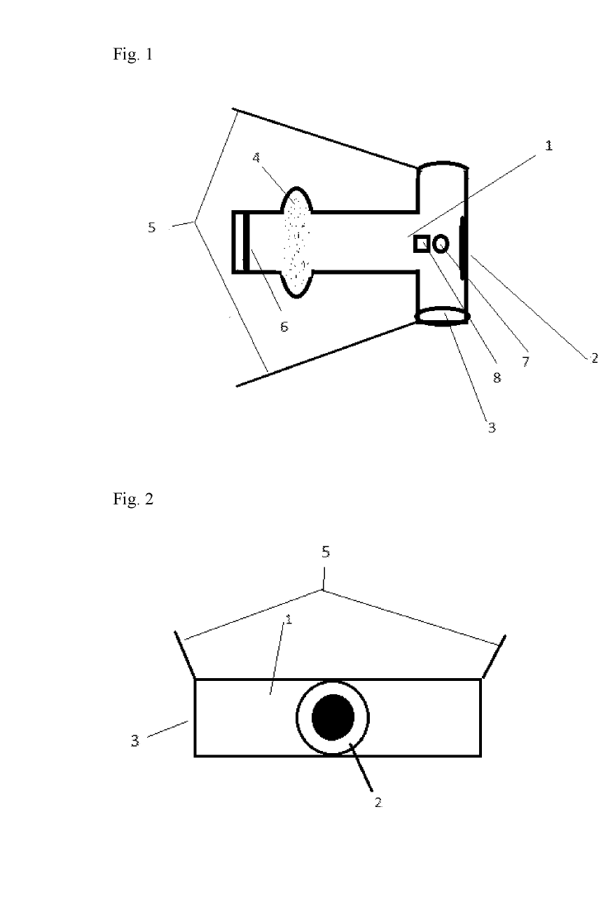

Referring now to the invention in more detail, in FIG. 1 (top perspective) and FIG. 2 (front end perspective), there is shown a multi-inlet mouth held filter apparatus 1, with a tubular housing for connection to the mouth and additional housings as filter inlets. While shown here as a two-inlet filter apparatus, the design is not limited to only two inlets, and other designs with more inlets can be used to allow for less conductance loss of airflow and thus easier breathing. The multi-inlet filter apparatus 1 has an optional replaceable one-way valve, also called a check valve, 2 for easier exhalation and moisture management, insertion slots 3 for filters, a flanged mouthpiece 4, made of rubber or other elastomeric material, to provide a seal against the environment using one's lips, with optional straps 5 to hold the filter apparatus to the face and a rubber (or other reversibly compressible material) indented section 6 to comfortably bite down on the filter apparatus to further hold the filter apparatus in place. Compared to current respirators which use the entire mask as a filter, the multi-inlet filter 1 reduces filter material area to reduce cost of the filtration apparatus while simplifying a proper seal by using the flexible mouthpiece 4, together increasing cost efficacy of the filter apparatus. An optional indicator 7 is either a multi-color LED light to indicate levels of pollution and required filter, or an LCD display to transmit information. An optional wireless communicator and power source 8 communicates with, without limitation, external sensors, computers, other wireless devices, or handheld or worn electronic devices, to receive or send information related to pollution, use of the apparatus, or potential health information.

In further detail, still referring to FIG. 1 and FIG. 2, the multi-inlet filter 1 can be made of plastic, rubber, metal, or other rigid or semi-rigid polymers. The mouthpiece 4 is held between the lips to ensure a seal so that air can only travel through the filters. The distance from the reversibly compressible indented section 6 to the flexible mouthpiece 4 is sufficient to allow varying clearances between the indented section 6 and mouthpiece 4. The size of flexible mouthpiece 4 varies based on the size of the user. The length of the filter apparatus from the rubber mouthpiece 4 to the filter insertion slots 3 is sufficient to allow small clearance from the lips to filter insertion slots 3. The length of the filter insertion slots 3 vary to fit the filters, based on the intended use of the filtration apparatus. The optional wireless communicator and power source 8 sends and receives information to and from a plurality of other devices and sources, including, without limitation, a central database that stores anonymous information about pollution in different locations and filter use, a smart phone, or any other external device. An indicator 7, with an LED for low-cost apparatuses, and an LCD for higher-cost apparatuses, receives information from the wireless communicator or an internal information source to indicate the type and level of filters necessary for ambient pollution, and when filters need replacement. The location of indicator 7 and wireless communicator and power source 8 is not fixed, but could be anywhere on the filter apparatus, including inside the apparatus.

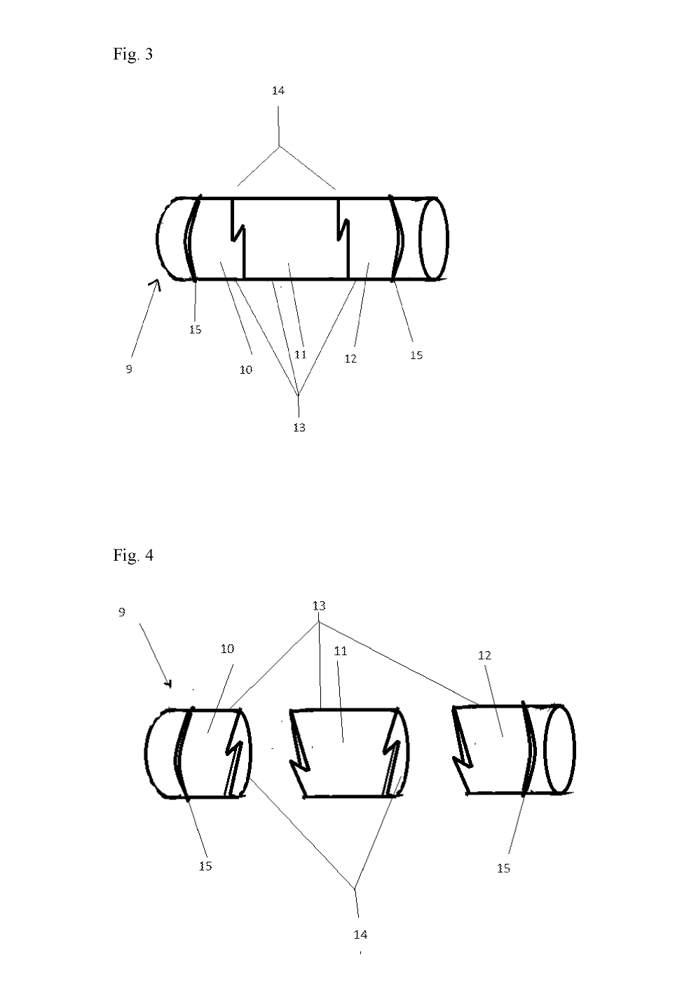

Referring now to FIG. 3 and FIG. 4, the multi-section replaceable filter 9 has multiple sections 10, 11, and 12 (or more or less, depending on the application; while three sections are used in this diagram, any number of sections can be brought together). Each filter section can house a separate filter type designed to filter different compounds or chemicals (air pollutants). Each filter section has a plastic, polymer, or metal outer shell 13 with a filter held inside, and each section filters different physical or chemical species. Each section can be individually replaced to tailor the multi-stage filter to the environment to reduce cost of the filter apparatus. For example, in an environment where filter section 10 is used frequently, but filter section 11 is rarely necessary, replacing filter section 10 when it expires, rather than replacing an entire filter apparatus, results in significant cost savings for the user. Filter sections lock together with complementary shaped edges 14. Complementary shaped edges 14 can be either the filter section housing or have rubber edges to ensure seal between filter sections. Filter sections at the ends of the filter have O-rings 15, on the outside to ensure that ambient air goes through the filter and not around it. In some embodiments, between each filter section is an optional pressure monitor to indicate when a filter is becoming clogged and needs replacement. The first filter in the series locks into the housing with a specific matching locking design built into the first filter and into the filter inlet housing of the filter apparatus. The filter(s) after the first filter lock(s) to the first filter, (or subsequent filters) and thus is or are also held in place by the lock between the first filter and the filter apparatus.

In an alternative embodiment, the filter 9 is multi-stage with several different filters, all of the filters being combined into a single section for ease of use if a user prefers not swapping individual sections.

In some embodiments, still referring to FIG. 3 and FIG. 4, the filter arrangement comprises, without limitation, one section with a microfiber filter, polyester filter, or other filter type to filter out PM2.5, which is particulate matter smaller than 2.5 microns produced by power plants and known to be carcinogenic, another section with an activated carbon fiber filter to filter ozone, which is a byproduct of power production and combustion engines, known to be a health hazard, and another section with pitch-activated carbon fibers to filter out NO₂ and SO₂, which are byproducts of power production and some combustion engines, known to cause long-term lung damage and asthma.

The PM2.5 filter, common to other respirators, differs in design in that this design of a long filter allows for a filter between 3-35 mm thickness, rather than the current thin filters, allowing the use of lower-cost materials to provide increased efficiency per unit cost. Additionally, the use of a graded particulate filter, with a coarse outer filter for larger particulates and a fine filter for PM2.5 allows more efficient filtering.

The SO₂ and NO₂ filter differs from other chemical filters in that it uses pitch-carbon, PAN carbon, or chemically enhanced carbon as the filter medium. Current carbon fiber filters are made from charcoal. Pitch carbon and polyacrylonitrile carbon (PAN-carbon), made from coal pitch, petroleum pitch, or tar pitch, is currently not used for filters, and, due it its expense, is used almost exclusively as a component for composites in airplane construction. The chemical properties of pitch/PAN carbon provide nearly double the adsorption surface area compared to standard activated carbon filters, and also have nitrogen inclusions which chemically enhance the catalyses and subsequent breakdown of SO₂ and NO₂. Another embodiment of this filter is a chemically enhanced activated carbon or pitch/PAN carbon fiber. Currently no pollution mask uses chemically enhanced carbon fibers to enhance filtration and reduce pressure drop for a given filtration efficacy. The filter according to the invention optionally has activated carbon or pitch carbon augmented with, without limitation, one or more of iron (Fe), copper (Cu), vanadium (V), or titanium dioxide (TiO₂) nanoparticles, as these elements attract SO₂ and NO₂, to chemically enhance filtration. These particles catalytically alter SO₂ and NO₂ to forms that are more readily sequestered in the filter. These can allow for a thinner filter for a given efficacy, reducing how much the filters restrict breathing.

All of the above described filter sections are removable if they are unnecessary for certain environments, or additional sections are added for more types of pollutants. Air flows through filter sections from first filter to last filter, which progressively scrubs harmful physical and chemical species (pollutants) and/or moisture from ambient air. Each type of filter is available in several grades of efficacy, allowing users to use less expensive and less restrictive filter grades when more expensive ones are not necessary. Each type of filter section is optionally available with differing colors and print to indicate the compounds or chemicals filtered, as well as the efficacy level of the replaceable filter section. Filter lengths may also vary by the substance filtered to further distinguish type of filtration. Each filter optionally has a color band associated with the strength of the filter, the color also being associated with the Air Quality Indicator in the country of sale or use, providing a user with a simple metric to understand which level of filter is needed.

Referring now to FIG. 1 through FIG. 4, a multi-stage filter 9 is inserted into filter insertion slots 3 (channels in the filter inlet housings, which are in fluid communication with the longitudinal channel in the first housing) on the filter apparatus 1. The filter 9 can be removed and taken apart to replace separate filter sections and reassembled and re-inserted into filter insertion slots 3.

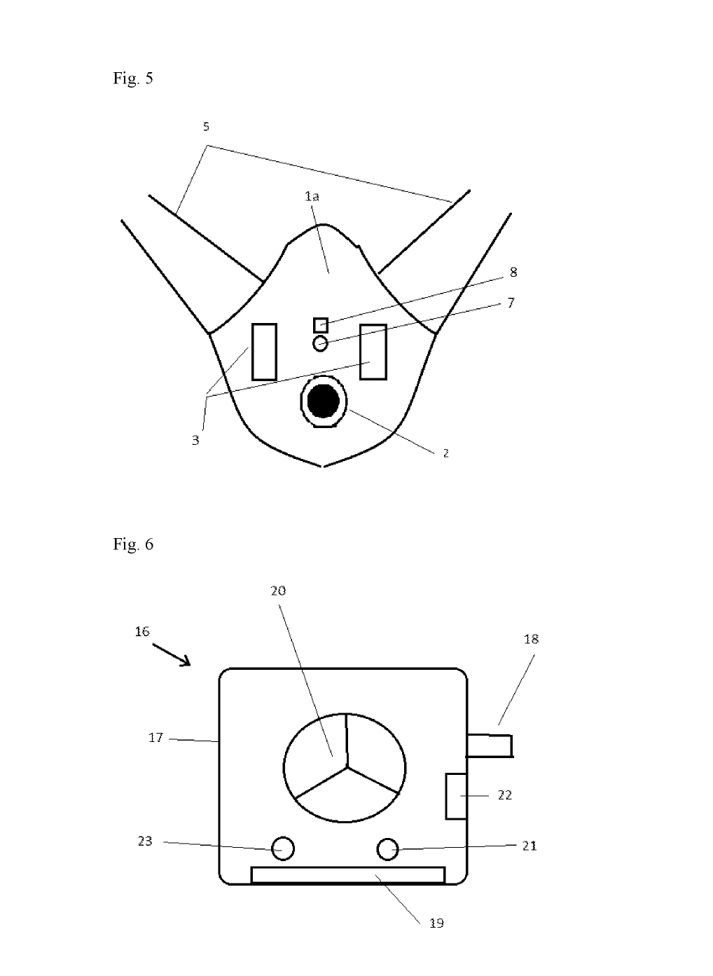

In some embodiments, referring now to FIG. 3 and FIG. 5, a mask air filtration apparatus 1a is used with the replaceable multi-stage filter 9. The mask air filtration apparatus 1a can be made from, without limitation, plastic, other polymers, metals, and other materials, with the material at the face edge of the mask being elastomeric, such that it can form to a face and create a good seal against the environment. Mask filtration apparatus 1a has an optional or replaceable one-way exhalation valve 2 to ease exhalation and reduce moisture buildup in the mask. A replaceable multi-stage filter 9 is inserted into filter insertion apertures or slots 3. The mask is held in place with straps 5. In some embodiments, an optional LED or LCD display 7 is either a multi-color LED light to indicate levels of pollution and required filter, or an LCD display to transmit information. In some embodiments, an optional wireless communicator and power source 8 communicates with, without limitation, external sensors, computers, other wireless devices, or handheld or worn electronic devices, to receive or send information related to pollution, use of the apparatus, or potential health information. In some embodiments, the mask air filtration apparatus further comprises a sensor to measure the pollution level (not shown). The location of LED or LCD 7, the wireless communicator and power source 8, and the pollution level sensor is not fixed, but can be anywhere on the mask, including inside the mask.

In some embodiments, referring now to FIG. 3 and FIG. 6, a fan and housing device 16 can be used with the multi-stage filter 9 to create a clean personal environment. A housing 17 has attachment or stand 18 to attach to baby strollers, cribs, or other furniture, or to rest on a surface. Replaceable filter holder 19 holds multi-stage filter 9 for the device. Fan or pump 20 pulls air through the filter and directs filtered air towards the user. Input port 21 allows for optional solar charging of the portable device. Pollution detector 22 detects pollution levels directly or contains a transmitter to connect to an external device such as a phone or other portable device to transmit pollution levels and other information. LED or LCD indicator 23 can change color or display text to indicate pollution levels, either measured within the device or transmitted into the device from an external source.

Among the advantages of the present invention is that it is portable, less expensive to manufacture and buy, less expensive to maintain than current filters, and more effective than most filters in filtering out more harmful species. The small design and materials chosen reduces the cost of the filter apparatus. The embodiments in which the multi-stage filter has each section being replaceable drastically reduces the cost of replacing filters by allowing the user to replace individual sections, rather than an entire filter. For portable filters, the filter apparatus and filter cost less to maintain than most respirators in that only the filter is replaced, not the entire mask. The mouth-held filter apparatus is more effective in that the multi-inlet filter apparatus guarantees a complete seal compared to difficult-to-seal masks, since there is no mask edge inconformity to break the seal, forcing air to go through the filter and ensuring breathing of filtered air. The portable filtration device that can connect to strollers, other furniture, or be used in one's personal space is more effective in that it is portable and can filter multiple harmful pollutants. The filter is more effective than nearly all other filters in that it filters particulate matter and in some embodiments chemical compounds and/or moisture, rather than just particulate matter. In preferred embodiments, the present invention is a personal filtration apparatus to filter out particulate matter and chemicals.

The following Examples are provided to further illustrate certain preferred embodiments of the invention and are not to be construed to limit the scope of the invention.

### Example 1

### Efficacy of a PM2.5 Filter

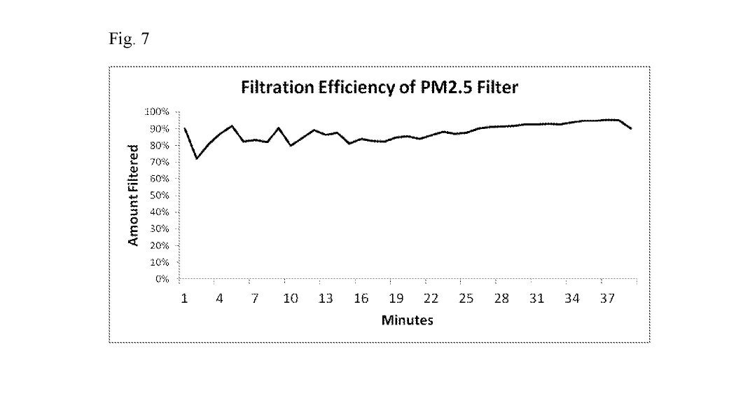

Referring to FIG. 3 and FIG. 4, in some embodiments, one of the replaceable filters 10, 11, and 12 will be a particulate matter filter, and another will be an SO₂ and NO₂ filter. In an embodiment of the invention, a PM2.5 particulate matter multi-layered polyester filter, with a first course layer for larger particles, and a fine layer with a Minimum Efficiency Reported Value (MERV) rating of 13 (out of 20) was used. The layers were stacked by gluing with a fine glue that does not block air flow. This filter showed a 90% efficacy of filtering harmful particulate matter. Dylos particle counters were used to determine particle matter pollution levels before and after the filter. Particulate matter pollution was measured simultaneously before and after the filter with two calibrated particle counters. The difference in particles counted indicates the efficacy of the filter. Shown in FIG. 7 are the results of the test, indicating a nearly 90% effective filtration of PM2.5

### Example 2

### Efficacy of an SO₂ Pitch Carbon Fiber Filter

Still referring to FIG. 3 and FIG. 4, and multi-part filters 10, 11, and 12, SO₂ filtration was tested using a single ply of unmodified pitch-based carbon fibers as a filter medium, and NO₂ and SO₂ filtration efficacy testing was tested on a woven polyacrylonitrile (PAN). Pitch based carbon is made from hydrocarbon pitch, rather than the activated charcoal typically used for activated carbon fibers for filtration. PAN based carbon is made from PAN that is carbonized. The filters in this trial averaged 9 mm in length and were woven into a fine mesh for the pitch filter, and a felt for the PAN filter. The data were obtained using a UV-Vis spectrometer and calibration gas containing 5 ppm SO₂ and 5 ppm NO₂ to determine SO₂ and NO₂ filter efficacy with the pitch based and PAN based carbon filters. SO₂ and NO₂ pollution was measured with and without exposure to the filter. The difference in SO₂ pollution levels indicate the efficacy of the filter. The results showed a 15% NO₂ and SO₂ filtration efficacy for a single ply pitch-based filter, and an 80% efficacy for a multi-ply PAN felt.

While the foregoing written description of the invention enables one of ordinary skill to make and use what is considered presently to be the best mode thereof, those of ordinary skill will understand and appreciate the existence of variations, combinations, and equivalents of the specific embodiment, method, and examples herein. The invention should therefore not be limited by the above described embodiment, method, and examples, but by all embodiments and methods within the scope and spirit of the invention as claimed.

What is claimed is:

1. A mouth-held multi-inlet air filter apparatus comprising a first tubular housing having a proximal end, a length and a distal end, wherein an indentation containing an elastomeric material is present at the proximal end to allow teeth to bite down on the apparatus and wherein a flanged and flexible mouthpiece is disposed distal to the indentation to allow lips of the mouth to form a seal with the apparatus, wherein a longitudinal channel is disposed within the housing, the channel being in fluid communication with the mouth and with a plurality of filter inlets, each filter inlet comprising an additional housing surrounding a channel having an identical replaceable filter disposed in an airtight manner therein, wherein each identical filter removes from air drawn through the filter in the filter channel and through the longitudinal channel into the mouth, one or more of PM2.5 particles, SO₂, NO₂, O₃ and water vapor.

2. A replaceable filter for an air filter apparatus, wherein the replaceable filter is a multi-section filter having different sections in series, and wherein different sections filter different pollutants.

3. The replaceable filter according to claim 2, wherein at least one filter section is a PM2.5 filter and at least one filter section filters SO₂ and NO₂, or O₃.

4. The replaceable filter according to claim 3, wherein at least one edge of each filter section is in operable contact with a pressure monitor to indicate when the filter section is becoming clogged and needs to be replaced.

5. The replaceable filter according to claim 2, wherein the air filter apparatus is a mouth-held multi-inlet air filter apparatus.

6. The replaceable filter according to claim 2, wherein the air filter apparatus is a respirator or gas mask.

7. The replaceable filter according to claim 2, wherein the filter apparatus is a portable filtration device that can attach to a baby stroller.

8. The replaceable filter according to claim 2, wherein the filter apparatus is a portable filtration device that can be used to filter air in a user's personal space

9. A mask air filtration apparatus comprising a mask with an elastomeric face edge to form a seal with a face, a one-way exhalation check valve, straps to hold the mask in place, a plurality of filter insertion apertures, an LED indicator for pollution levels by changing color and, disposed within the filter insertion apertures in an airtight manner, identical multi-stage filters according to claim 2, 3, 4, 5, or 6.

10. The mask air filtration apparatus according to claim 9, further comprising a wireless communicator and power source that communicates with one or more of external sensors, computers, other wireless devices, or handheld or worn electronic devices, to receive or send information related to pollution, use of the apparatus, or health information.

11. An air filtration device for a baby stroller comprising a plastic housing with a fan and filter apparatus, disposed within the filter insertion apertures in an airtight manner, identical multi-stage filters according to claim 2, 3, 4, 7, or 8.

12. The portable air filtration device according to claim 11, further comprising a sensor to measure levels of pollution and an LED light source to indicate pollution levels by changing color.

13. The air filtration enclosure device according to claim 11, further comprising a wireless communicator and power source that communicates with one or more of external sensors, computers, other wireless devices, or handheld or worn electronic devices, to receive or send information related to pollution, use of the apparatus, or health information.

14. The air filter according to claim 2, wherein the SO₂ and NO₂ filter comprises one or more of activated carbon, pitch carbon, and PAN carbon.

15. The air filter apparatus according to claim 14, wherein the one or more of activated carbon, pitch carbon, and PAN carbon is augmented by one or more of iron (Fe), copper (Cu), vanadium (V), or titanium dioxide (TiO₂) nanoparticles.

16. An air filter comprising of one or more of pitch carbon and PAN carbon.

17. The air filter according to claim 16, wherein the pitch/PAN carbon is augmented by one or more of iron (Fe), copper (Cu), vanadium (V), or titanium dioxide (TiO₂) nanoparticles.

18. A stroller, baby carrier or other furniture comprising the air filtration enclosure device according to claim 11.

19. The air filtration enclosure device according to claim 11 or 18, further comprising a pneumatic pump or fan to force air through the air filter.
